(19) United States
(12) Patent Application Publication
(10) Pub. No.: US 2010/0183754 A1
(43) Pub. Date: Jul. 22, 2010

(54) Extract and compound for blood pressure regulation

(75) Inventors: Wen-Chi Hou, Taipei City (TW); Yeh-Lin Lu, Taoyuan County (TW); Yin-Shiou Lin, Taipei County (TW); Chi-Luan Wen, Taichung County (TW); Lih-Geeng Chen, Keelung City (TW)

Correspondence Address: BACON & THOMAS, PLLC, 625 SLATERS LANE, FOURTH FLOOR, ALEXANDRIA, VA, 22314-1176, US

(73) Assignee: Taipei Medical University, Taipei City (TW)

(21) Appl. No.: 12/461,402

(22) Filed: Aug. 11, 2009

(30) Foreign Application Priority Data
Jan. 22, 2009 (TW) 98102503

(51) Int. Cl. A61K 36/87

(52) U.S. Cl. 424/766

(57) ABSTRACT

The present invention provides an extract of Taiwanese wild grape (Vitis thunbergii var. taiwaniana), which is prepared by extracting one or more tissues of Vitis thunbergii var. taiwaniana with 10 wt % to 100 wt % alcohol solution or water to obtain an extract solution, and then drying said extract solution. The extract of Vitis thunbergii var. taiwaniana of the present invention has the function of regulating blood pressure, and therefore can be used to prevent and treat hypertension. The present invention also provides a method for purifying (+)-vitisin A and ampelopsin C from Vitis thunbergii var. taiwaniana and a use of these two compounds for hypertension prevention and treatment.

### BACKGROUND OF THE INVENTION

1. Field of the Invention

The present invention relates to an extract of Taiwanese wild grape (Vitis thunbergii var. taiwaniana), and more particularly to an extract of Taiwanese wild grape which can be used to prevent and treat hypertension.

2. Description of the Related Art

Hypertension, also known as high blood pressure, recently becomes one of the ten leading causes of death and one of the most important risk factors of coronary artery diseases (CADs) and cerebral vessels diseases. Hypertension is a very common cardiovascular disease, and its symptoms only occurred at the late-stage of the progression. The patients who suffered from hypertension for a long time intend to have complications of organ injury, including heart hypertrophy, heart failure, coronary arteriosclerosis, stroke, aortic aneurysm, renal vascular disease, retinopathy and vision loss.

Administration of antihypertensive drugs is the main therapy of hypertension. However, day-to-day health care guidelines, such as weight-control, moderate exercise, emotion management, quit smoking and proper diet are also effective for preventing hypertension. For proper diet, reducing salt and fat contents in foods or uptaking enough potassium, magnesium and calcium are useful for controlling blood pressure.

According to recent researches, some peptides obtained from natural animal or vegetable foods have the function of inhibiting the activity of angiotensin I-converting enzyme (ACE), resulting in reducing the formation of angiotensin II; therefore, these peptides can be used to regulate blood pressure. Researchers had isolated peptides with the function of reducing blood pressure from food proteins. These peptides are called angiotensin converting enzyme inhibitors (ACEIs), which can effectively inhibit increasing blood pressure. These food-isolated peptides may have different amino acid sequences and different lengths, but they have similar functions. The biggest difference of the ACEIs isolated from foods and the chemically synthesized drugs is that those chemically synthesized drugs have very strong pharmaceutical effects, however, ACEIs isolated from foods have milder pharmaceutical effects with fewer side effects. Therefore, the ACEIs isolated from food are more suitable to be used as daily healthy foods to prevent hypertension. After hydrolysis by various enzymes, the ACEIs obtained from natural foods are cut into peptide fragments with different lengths, wherein some short peptides thereof have a very strong ACE inhibiting effect. Today, the scholars have found that ACEIs can also be isolated from many fermented foods, such as sake and sake lees (Saito et al., 1994a,b), soy sauce (Kinoshita et al., 1993) and fermented milk products (Nakamura et al., 1995a, b). Besides proteins and peptides, Park et al. (2003) have indicated that chitosans with different deacetylation levels have different ACE inhibiting activities, and this shows that specific polysaccharides also have ACE inhibiting activities. Moreover, pomegranate juice (Aviram and Dornfeld, 2001), flavan-3-ols and procyanidins (Actis-Goretta et al., 2003), tannins (Liu et al., 2003) and the structure analogues of myricetin galloylglycoside (Lee et al., 2006) also have ACE inhibiting activities.

Vitis thunbergii var. taiwaniana is an endemic species in Taiwan. Taiwan Endemic Species Research Institute has cataloged Vitis thunbergii var. taiwaniana as a Taiwan original medicinal plant. In Chinese Folk Medicine, the roots and leaves of Vitis thunbergii var. taiwaniana have been used to clear heat, dispel dampness, induce diuresis, detoxify, alleviate edema, stop bleeding and promote tissue regeneration; and, its stems and branches have been used to dispel wind, eliminate dampness, detoxify and alleviate edema. Vitis thunbergii var. taiwaniana is a general Chinese medicine in Taiwan.

In view of this, the Vitis thunbergii var. taiwaniana extract of the present invention is used for controlling blood pressure, and the extract have a better pharmaceutical effect and it is suitable for long-term blood pressure regulation.

### SUMMARY OF THE INVENTION

One object of the present invention is to provide a Vitis thunbergii var. taiwaniana extract. The Vitis thunbergii var. taiwaniana extract contains compounds having ACE inhibiting activity, which can be used to regulate blood pressure, and therefore can be used in the treatment of hypertension, and especially suitable to be used as daily healthy foods for preventing hypertension.

According to one aspect of the present invention, the Vitis thunbergii var. taiwaniana extract provided by the present invention is prepared by the following steps: extracting one or more tissues of Vitis thunbergii var. taiwaniana with 10 wt % to 100 wt % of alcohol aqua solution or water to obtain an extract solution, and then drying said extract solution.

Preferably, said alcohol is methanol, ethanol, propanol or isopropanol. More preferably, said alcohol is 50 wt % or more of alcohol solution; said water is boiling water.

Preferably, said extract is extracted with water, and said extract solution is further processed by the following steps: gradient-eluting said extract solution by using porous adsorbent resin as stationary phase and water to alcohol as mobile phase; and then collecting eluates eluted by 50 wt % or more of alcohol solution.

Preferably, said one or more tissues of Vitis thunbergii var. taiwaniana is root or stem.

According to another aspect of the present invention, the present invention provides a use of the above-mentioned Vitis thunbergii var. taiwaniana extract of the present invention in preparing a healthy food or functional food for blood pressure regulation.

According to another aspect of the present invention, the present invention also provides a pharmaceutical composition for hypertension prevention or treatment, which contains an effective amount of the above-mentioned Vitis thunbergii var. taiwaniana extract of the present invention.

According to another aspect of the present invention, the present invention further provides a method for isolating (+)-vitisin A or ampelopsin C from Vitis thunbergii var. taiwaniana, which comprises the steps of: (a) dissolving the above-mentioned Vitis thunbergii var. taiwaniana extract of the present invention in the alcohol solution to obtain a dissolved solution; (b) gradient-eluting the dissolved solution from step (a) by using dextran gel (ex. Sephadex) as stationary phase and an alcohol to a ketone as mobile phase, and then collecting first batch of eluates; (c) gradient-eluting the first batch of eluates from step (b) by using amorphous silicon as stationary phase and a C1 to C3 organic acid to acetonitrile as mobile phase, and then collecting second batch of eluates; and (d) identifying (+)-vitisin A or ampelopsin C in the second batch of eluates obtained from step (c).

Preferably, said alcohol is methanol, ethanol, propanol or isopropanol; said ketone is acetone; and said C1 to C3 organic acid is trifluoroacetic acid.

According to another aspect of the present invention, the present invention also provides a use of (+)-vitisin A or ampelopsin C for hypertension prevention or treatment.

According to another aspect of the present invention, the present invention further provides a product for hypertension prevention or treatment, which contains one or more tissues of Vitis thunbergii var. taiwaniana, a Vitis thunbergii var. taiwaniana extract, (+)-vitisin A or ampelopsin C.

Preferably, the type of said product is in form of drink, tablet or instant drink powder.

To sum up, the Vitis thunbergii var. taiwaniana extract provided by the present invention contains compounds having ACE inhibiting activity, and therefore can be used to reduce blood pressure. Compared with the medicines now generally used for hypertension, the Vitis tbunbeigii var. taiwaniana extract of the present invention has a milder pharmaceutical effect, and fewer side effects, which is suitable to be used as daily healthy foods for preventing hypertension.

### BRIEF DESCRIPTION OF THE DRAWINGS

The object and advantages of the present invention will become apparent to those ordinarily skilled in the art after reviewing the following detailed descriptions and accompanying drawings, in which:

FIG. 1A exhibits the tissue culture plantlet of Vitis thunbergii var. taiwaniana.

FIG. 1B shows the tissue culture plantlet of Vitis thunbergii var. taiwaniana cultured by four different plant growth substrates.

FIG. 2A shows the Vitis thunbergii var. taiwaniana cultivated in the field, which were transferred from tissue culture plantlets; and FIG. 2B shows the Vitis thunbergii var. taiwaniana harvested after field cultivation for 5 to 6 months.

FIG. 3A shows the morphology of the branches and leaves of the Vitis thunbergii var. taiwaniana after field cultivation for several years.

FIG. 3B shows the morphology of the roots of the Vitis thunbergii var. taiwaniana after field cultivation for several years.

FIG. 4 illustrates the experimental result of the ACE inhibiting activity of the Vitis thunbergii var. taiwaniana extract obtained in example 1.

FIG. 5 shows the ACE inhibition activity of various concentrations of the extracts obtained in example 1.

FIG. 6 illustrates the experimental result of the ACE inhibiting activity of hot water extract of Vitis thunbeigii var. taiwaniana in tea powder form obtained in example 2.

FIG. 7 illustrates the 24-hour blood pressure test result of SHRs after feeding with the hot water extract of the root and stem of Vitis thunbergii var. taiwaniana cultivated in the field, which is transferred from tissue culture plantlets obtained in example 1.

FIG. 8 shows the blood pressure of the rats long-term fed with the Vitis thunbergii var. taiwaniana extract obtained in example 1.

FIG. 9 illustrates the flow chart for isolating components from Vitis thunbergii var. taiwaniana root as described in example 5.

FIG. 10 illustrates the flow chart for isolating components from Vitis thunbergii var. taiwaniana stem as described in example 5.

FIG. 11 shows the result of ACE inhibiting activity of various concentrations of (+)-vitisin A and ampelopsin C purified in example 5; wherein, (+)-vitisin A is marked as VTT-1; ampelopsin C is marked as VTT-2.

FIG. 12 shows the 24-hour blood pressure test result of SHRs after feeding with (+)-vitisin A purified in example 5; wherein, (+)-vitisin A is marked as VTT-1.

### DETAILED DESCRIPTION OF THE PREFERRED EMBODIMENT

As described above, the Vitis thunbergii var. taiwaniana extract provided by the present invention can be used to prevent or treat hypertension.

One of the important technical features of the present invention is that the extract obtained by extracting Vitis thunbergii var. taiwaniana with an alcohol aqueous solution or water have ACE inhibiting activity. Preferably, said alcohol aqueous solution contains 50% or more of alcohol; and said water is boiling water.

With reference to the following disclosures combined with the accompanying embodiments and drawings, the advantages and technical features of the present invention are illustrated and understood. Various modifications and alterations can be made without departing from the spirits of the present invention and the scope of the invention is defined by the appended claims.

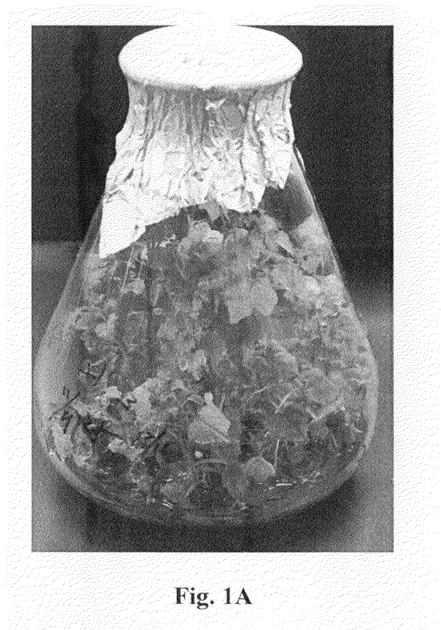

FIGS. 1A and 1B show the tissue culture plantlets of tissue-cultured Vitis thunbergii var. taiwaniana. FIG. 1B shows the tissue culture plantlets of Vitis thunbergii var. taiwaniana cultured by four different plant growth substrates, wherein the plant growth substrates are IAA (indole-3-acetic acid) for TC-1; IBA (indole-3-butyric acid) for TC-2; CK (cytokinins) for TC-3; and NAA (naphthaleneacetic acid) for TC-4.

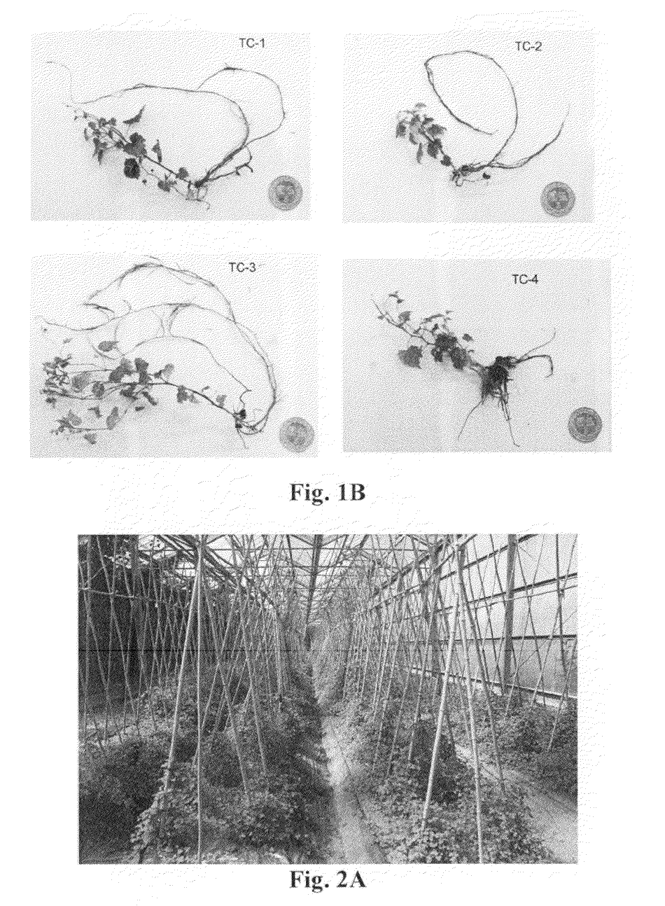

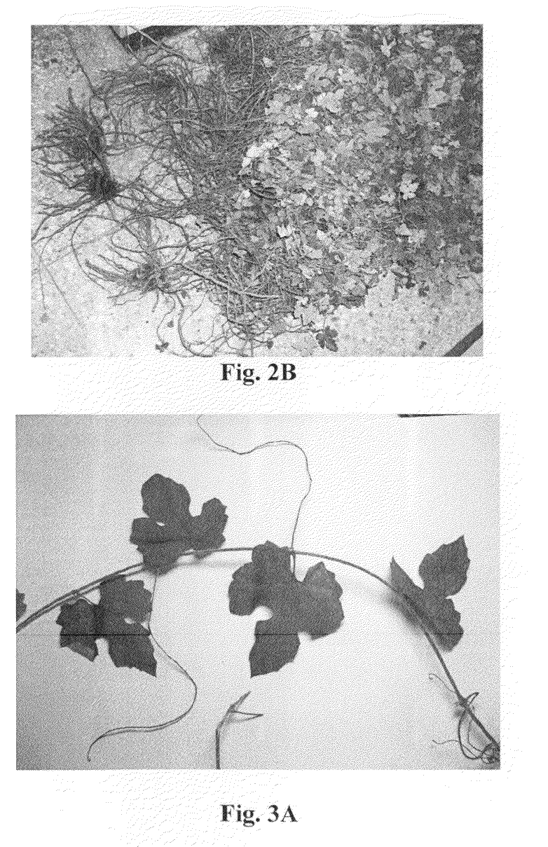

FIG. 2A shows the Vitis thunbergii var. taiwaniana cultivated in the field, which were transferred from tissue culture plantlets. FIG. 2B is the Vitis thunbergii var. taiwaniana harvested after field cultivation for 5 to 6 months. FIGS. 3A and 3B shows the morphology of the branches, leaves and roots of Vitis thunbergii var. taiwaniana after field cultivation for several years.

In the following examples, the above three types of Vitis thunbergii var. taiwaniana were extracted and tested.

### Example 1

### Extracting the Active Components of Vitis thunbergii var. taiwaniana

The plants to be used in the following experiment were confirmed as Vitis thunbergii var. taiwaniana by genetic identification. Various parts of fresh Vitis thunbergii var. taiwaniana were used as raw materials and they were air-blowing-dried by oven at 40° C. to obtain dried raw materials. A small amount of stems of Vitis thunbergii var. taiwaniana were extracted by heat reflux extraction, using methanol or ethanol as the extracting solvent. The extracts were then analyzed by HPLC (High Performance Liquid Chromatography). It was found that the components comprised in the extracts obtained from methanol and ethanol and their concentrations were almost the same (data not shown). Therefore, 50 wt % or more of methanol solution was used to extract and isolate the components of various parts of Vitis thunbergii var. taiwaniana. Table 1 lists the materials used and their corresponding labels. In the present embodiment, two types of Vitis thunbergii var. taiwaniana were employed: one was Vitis thunbergii var. taiwaniana cultivated in field for several years, in which its stems, leaves, branches and roots were used; and the other one was the tissue culture plantlets of Vitis thunbergii var. taiwaniana cultured by different plant growth substrates; said plant growth substrates used are as aforementioned: IAA for TC-1; IBA for TC-2; CK for TC-3; NAA for TC-4; as shown in Table 1.

TABLE 1

| | | |
|---|---|---|
| Vitis thunbergii var. taiwaniana | Stems | S |
| cultivation in field | Leaves | L |
| | Branches | B |
| | roots | R |
| Tissue culture plantlets of | TC-1 | TC-1R (roots) |
| Vitis thunbergii var. taiwaniana | | TC-1L (stems and leaves) |
| cultured by different plant | TC-2 | TC-2R (roots) |
| growth substrates | | TC-2L (stems and leaves) |
| | TC-3 | TC-3R (roots) |
| | | TC-3L (stems and leaves) |
| | TC-4 | TC-4R (roots) |
| | | TC-4L (stems and leaves) |

Moreover, these raw materials of Vitis thunbergii var. taiwaniana also could be extracted by hot water. The detailed process is exemplified as followings: the tissue culture plantlets of Vitis thunbergii var. taiwaniana were transferred in a field and cultivated for 5 to 6 months, and then their roots and stems were harvested. Each kilogram of raw materials were extracted by 10 times amount of water for 1 hour by heat reflux extraction. After that, the residues were extracted again by 10 times amount of water. Both of the extracts were combined, filtrated and dried. The dried product was referred as the hot water extract of Vitis thunbergii var. taiwaniana.

### Example 2

### Hot water extract of Vitis thunbergii var. taiwaniana in form of tea powder

For convenience, Vitis thunbergii var. taiwaniana also can be made as tea powder, which will be soaked in hot water to obtain the extract of Vitis thunbergii var. taiwaniana of the present invention. The detailed process is exemplified as followings: several tissue culture plantlets of Vitis thunbergii var. taiwaniana were washed by DD-water (deionized and distilled water), and then dried and weighted. The dried plants were cut into pieces and placed into tea bags, and then the tea bags were sealed. In other words, these pieces of Vitis thunbergii var. taiwaniana were in form of tea powder. The Vitis thunbergii var. taiwaniana tea powder was extracted twice by 100° C. hot water in the ratio of 1/10 (w/v) for 30 minutes. After being filtered, the filtrates were collected and then lyophilized for further uses. The lyophilized products were referred as the hot water extract of Vitis thunbergii var. taiwaniana tea powder.

### Example 3

### In Vitro ACE Inhibiting Assay

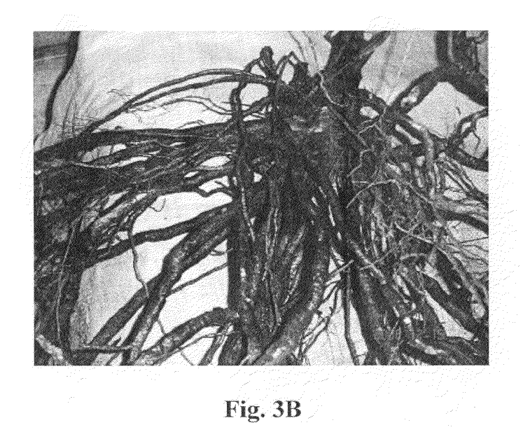

The ACE inhibiting activities of the Vitis thunbergii var. taiwaniana extracts obtained in example 1 is showed in FIG. 4. As shown in FIG. 4, by using the same weight (200 μg) of raw materials, the extracts of roots (R) and stems (S) from the plants cultivated in field have the highest inhibiting activities, while the extracts of leaves (L) and branches (B) from the same plants have lower inhibiting activities. For the extracts of tissue culture plantlets, the extracts of the roots from four tissue culture plantlets (TC-1R, TC-2R, TC-3R, TC-4R) have better inhibiting activities, which is obviously higher than the extracts obtained from the stems and leaves thereof (TC-1L, TC-2L, TC-3L, TC-4L).

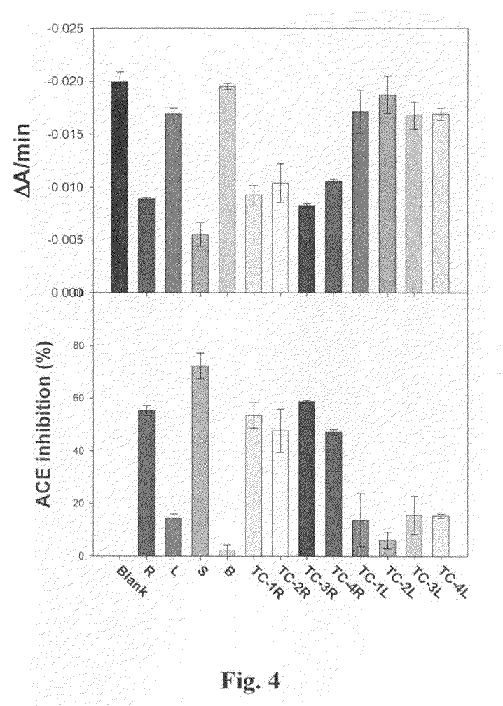

FIG. 5 shows the ACE inhibition of various concentrations of the extracts obtained in example 1. The half maximal inhibitory concentration (IC₅₀) for inhibiting 20 mU ACE are: R (136.61 μg/mL); S (69.49 μg/mL); TC-1 (98.87 μg/mL); TC-2 (132.05 μg/mL); TC-3 (99.04 μg/mL); TC-4 (102.46 μg/mL), respectively. Wherein, the data of TC-1 to TC-4 are obtained from the whole plant extracts of Vitis thunbergii var. taiwaniana cultured by various plant growth substrates, respectively.

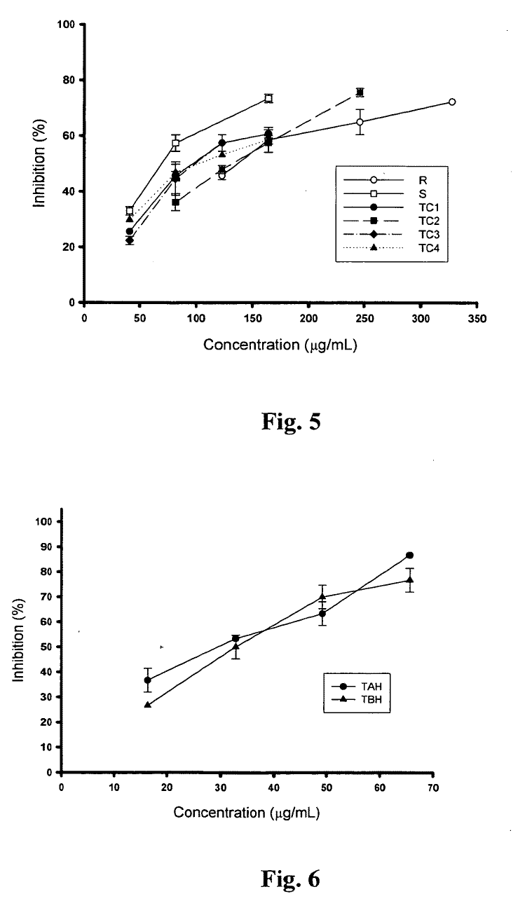

Moreover, the hot water extract of the Vitis thunbergii var. taiwaniana tea powder obtained in example 2 is used to perform the same ACE inhibiting assay, as shown in FIG. 6. The result shows that the hot water extract of the Vitis thunbergii var. taiwaniana tea powder also has ACE inhibiting activity. Wherein TAH represents the hot water extract of the whole plant tissue culture plantlet TC-1 cultured by the following method A, and the IC₅₀ thereof is 29.51 μg/ml; TBH represents the hot water extract of the whole plant tissue culture plantlet TC-1 cultured by the following method B, and the IC₅₀ thereof is 32.79 μg/mL. Method A: culturing the tissue culture plantlet for 30 days, replacing the old culture medium with new culture medium, and then culturing the plantlet for another 60 days; in which the plantlet were cultured for 90 days. Method B: culturing the tissue culture plantlet for 30 days, directly adding the new culture medium onto the old culture medium, and then culturing for another 60 days; in which the plantlet were cultured for 90 days.

### Example 4

### In Vivo Activity Analysis of the Vitis thunbergii var. taiwaniana Extract

The Vitis thunbergii var. taiwaniana extracts of the present invention were used in the in vivo blood pressure regulation assay. The present embodiment was performed on 24 heads of five- to six-week old male spontaneous hypertensive rats (SHRs). The rats were housed for 7 weeks and then used for the assay while they were about twelve- to thirteen-week old. The SHRs were randomly divided into experimental group (12 heads of SHRs) and control group (12 heads of SHRs). The rats of the experimental group were fed with the Vitis thunbergii var. taiwaniana extract obtained in example 1, and the rats of the control group were fed with distilled water for instead. After oral administration by feeding tube, the blood pressure of all SHRs was measured for a certain time interval. Table 2 shows the blood pressure changes of SHRs in the period of 24 hours after single oral administration of S (the extract of Vitis thunbergii var. taiwaniana cultivated in field) and TC-1 R in the dose of 20 mg/kg.

TABLE 2

| time | Δ SBP (mmHg) 20 mg/kg | | Δ DBP (mmHg) 20 mg/kg | |
|---|---|---|---|---|
| (hour) | S | TC-1R | S | TC-1R |
| 2 | −15.1 ± 6.6 | −14.1 ± 2.9 | −16.2 ± 7.3 | −11.1 ± 4.6 |
| 4 | −19.7 ± 5.6 | −16.3 ± 6.8 | −16.9 ± 5.5 | −17.7 ± 5.7 |
| 6 | −16.2 ± 2.1 | −9.1 ± 5.3 | −11.2 ± 3.5 | −11.9 ± 4.4 |
| 24 | −15.0 ± 3.5 | −10.9 ± 7.0 | −15.0 ± 2.5 | −6.7 ± 6.0 |

Δ SBP: systolic blood pressure changes
Δ DBP: diastolic blood pressure changes

As shown in Table 2, the blood pressure of the rats fed with the extract S obtained from Vitis thunbergii var. taiwaniana cultivated in field reached the lowest value at the 4^{th} hour, wherein the systolic blood pressure (SBP) and diastolic blood pressure (DBP) had decreased 19.7 mmHg and 16.9 mmHg, respectively. And, after 24 hours, the decrease levels of the SBP and DBP still maintained at 15 mmHg. The blood pressure of the rats fed with the extract TC-1R obtained from Vitis thunbergii var. taiwaniana tissue culture plantlet reached the lowest value at the 4^{th} hour, wherein the decrease levels of the SBP and DBP had decreased 16.3 mmHg and 17.7 mmHg, respectively. And, after 24 hours, the decrease level of SBP still maintained at 10.9 mmHg.

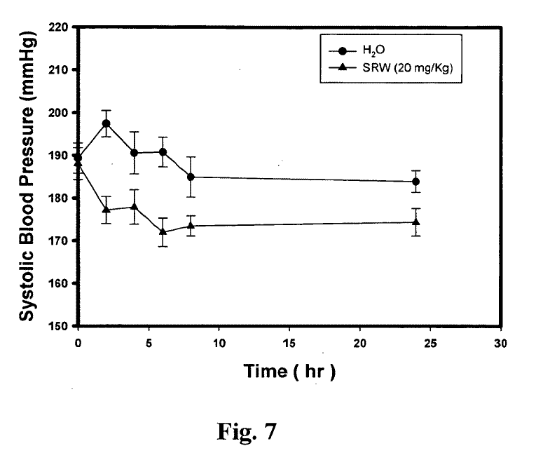

Similarly, the hot water extract of the roots and stems of the Vitis thunbergii var. taiwaniana cultivated in field for 5 to 6 months, which was transferred from tissue culture plantlets, was used in a 24-hour blood pressure test, as shown in FIG. 7 (the rats of the control group were fed with distilled water). As shown in FIG. 7, the blood pressure of SHRs in the experimental group (marked as SRW in FIG. 7) is significantly reduced within 24 hours.

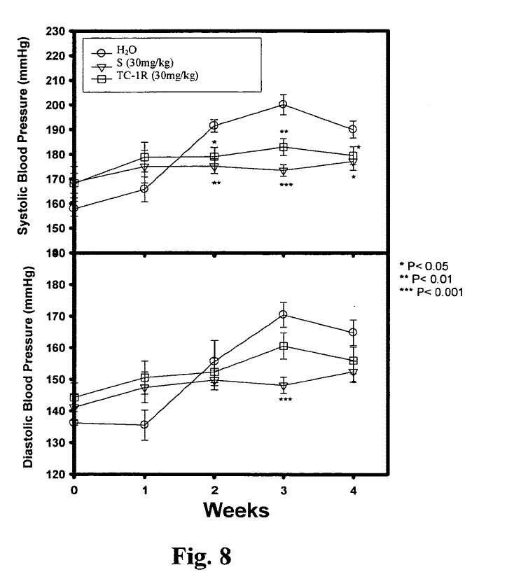

Also, the Vitis thunbergii var. taiwaniana extract obtained in example 1 was used in a long-term feeding test. The rats were orally administered once a day (30 mg/kg, SHR), and the blood pressure thereof was measured every week at a fixed time. FIG. 8 shows that both the stem extract of the plant cultivated in field for years and the root extract of tissue culture plantlet of Vitis thunbergii var. taiwaniana can reduce the blood pressure of SHRs at the 2^{nd} week after feeding (both SBP and DBP; the stars represent that there are significant difference (*, P<0.05; **, P<0.01) between the experimental group and control group at the same time).

### Example 5

### Purification of the Active Components of Vitis thunbergii var. taiwaniana

### Isolation of the Components of Root

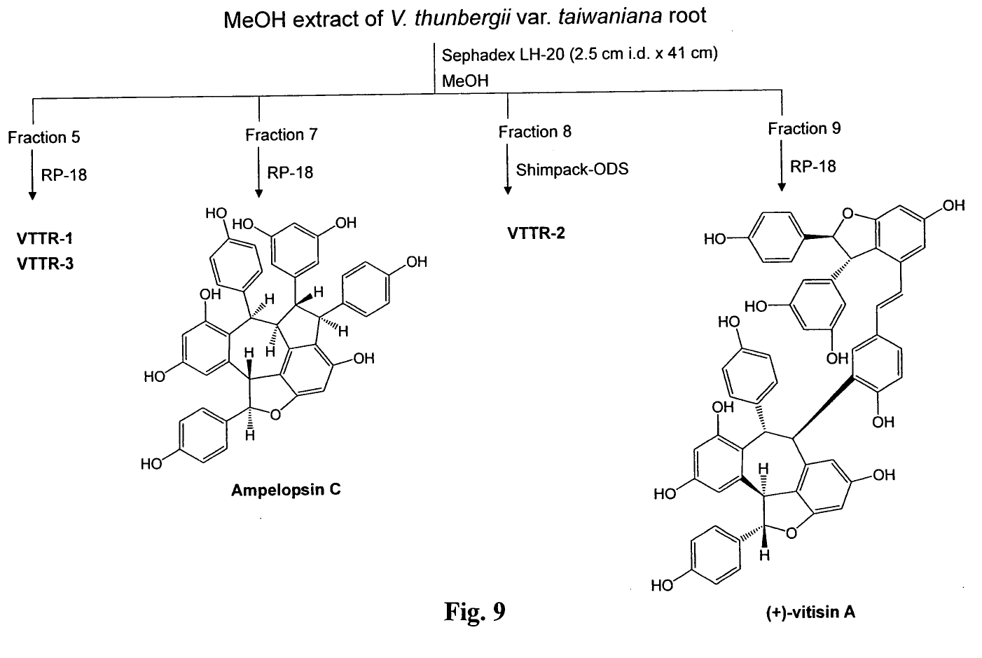

10 g of the methanol extract of Vitis thunbergii var. taiwaniana root obtained in example 1 was dissolved into methanol, and then subjected to chromatography by using Sephadex LH-20 gel column (2.5 cm i.d.×41 cm), in which methanol to 70% acetone as mobile phase, to separate the total components (comprised in elutes) into 10 fractions. The yield of each fraction was too low to be used for further activity examination. Therefore, the 9^{th} fraction was separated by using LiChroprep RP-18 (2.5 cm i.d.×48 cm) column and 0.05% TFA-CH₃CN (70:30) as mobile phase to obtain (+)-vitisin A (84.7 mg). The 7^{th} fraction was separated by using LiChroprep RP-18 (1.5 cm i.d.×36.5 cm) column and 0.05% TFA-CH₃CN (65:35) as mobile phase to obtain ampelopsin C (292.5 mg). The 5^{th} fraction was separated by using LiChroprep RP-18 (2.5 cm i.d.×56 cm) column and 0.05% TFA-CH₃CN (81:19) as mobile phase to obtain VTTR-1 (299.9 mg) and VTTR-3 (68 mg). And, the 8^{th} fraction was separated by using Shimpack-ODS (2.0 cm i.d.×25 cm) preparative HPLC column and 0.05% TFA-CH₃CN (65:35) as mobile phase to obtain VTTR-2 (28.5 mg). The structures of ampelopsin C and (+)-vitisin A were identified by comparing the NMR and MS spectra obtained in the experiment and that recorded in scientific literatures. VTTR-1, VTTR-2 and VTTR-3 were analyzed by NMR. The complete flow chart is shown as FIG. 9. The structures of ampelopsin C and (+)-vitisin A are shown as followings:

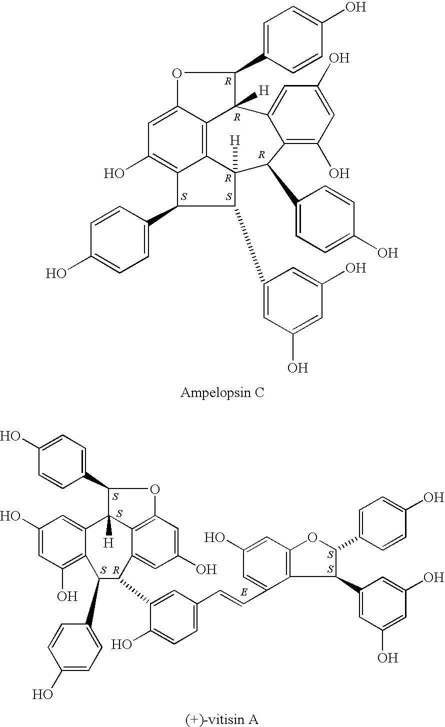

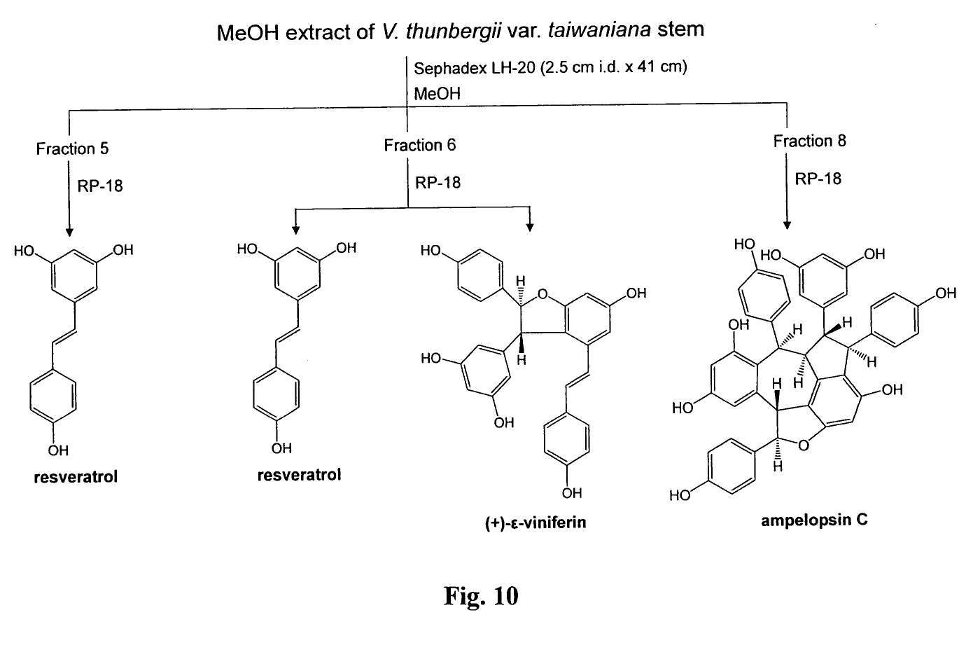

Isolation of the Components of Stem 8.85 g of the methanol extract of Vitis thunbergii var. taiwaniana root obtained in example 1 was dissolved into methanol, and then subjected to chromatography by using Sephadex LH-20 gel column, in which methanol to 70% acetone were used as mobile phase, to separate the total components (comprised in elutes) into 9 fractions. The 8^{th} fraction was separated by using LiChroprep RP-18 (2.5 cm i.d.×57 cm) column and 0.05% TFA-CH₃CN (70:30) as mobile phase to obtain ampelopsin C (110.1 mg). The 5^{th} fraction was separated by using LiChroprep RP-18 (2.5 cm i.d.×57 cm) column and 0.05% TFA-CH₃CN (75:25) as mobile phase to obtain resveratrol (50.1 mg). The 6^{th} fraction was separated by using LiChroprep RP-18 (2.5 cm i.d.×57 cm) column and 0.05% TFA-CH₃CN (75:25→>75:30) as mobile phase to obtain resveratrol (10.6 mg) and (+)-ε-viniferin (143 mg). The structures of resveratrol and (+)-ε-viniferin were identified by comparing the NMR and MS spectra obtained in the experiment and that recorded in scientific literatures. The complete flow chart is shown as FIG. 10.

### Example 6

### Activity Analysis for (+)-Vitisin A and Ampelopsin C

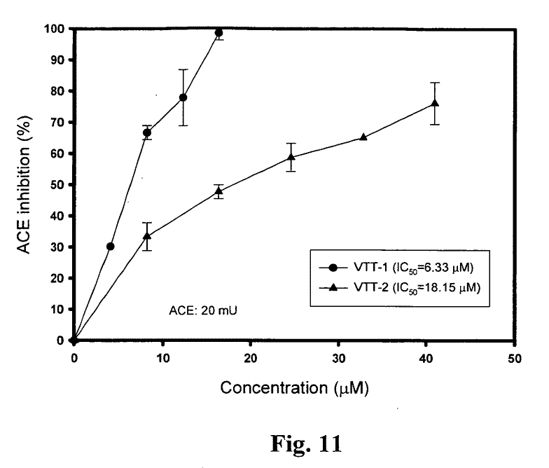

Various concentration of (+)-vitisin A and ampelopsin C purified in example 5 were used to perform ACE inhibiting activity assay. As shown in FIG. 11, (+)-vitisin A is marked as VTT-1; ampelopsin C is marked as VTT-2. FIG. 11 shows that (+)-vitisin A and ampelopsin C obviously have ACE inhibiting activity, wherein the IC₅₀ are 6.33 M and 18.15 μM, respectively.

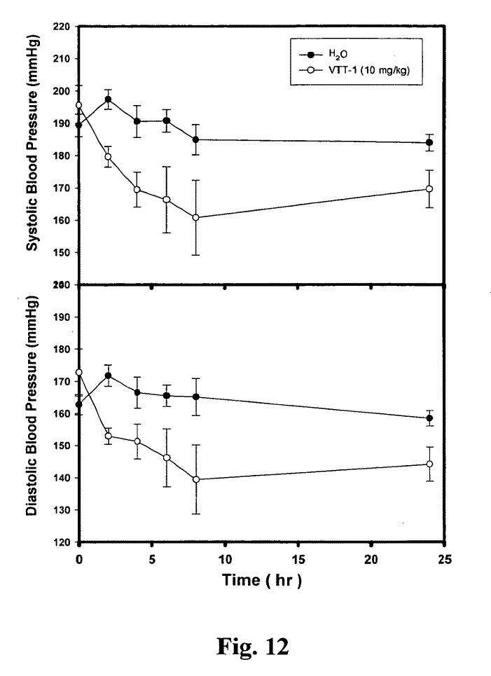

Moreover, the blood pressure of the SHRs fed with (+)-vitisin A purified in example 5 was measured during 24 hours after single oral administration. As shown in FIG. 12, the rats of experimental group were orally administered of (+)-vitisin A in the dose of 10 mg/kg (marked as VTT-1), and the rats of control group were fed with distilled water for instead. FIG. 12 shows that the blood pressure of the experimental group rats decreases significantly in the period of 24 hours after single oral administration with (+)-vitisin A.

To sum up, the effect of Vitis thunbergii var. taiwaniana of present invention for regulating blood pressure is mainly came from (+)-vitisin A and ampelopsin C.

### Other Embodiments

While the invention has been described in terms of what are presently considered to be the most practical and preferred embodiments, it is to be understood that the invention need not be limited to the disclosed embodiments. On the contrary, it is intended to cover various modifications and similar arrangements included within the spirit and scope of the appended claims, which are to be accorded with the broadest interpretation so as to encompass all such modifications and similar structures. Therefore, the above description and illustration should not be taken as limiting the scope of the present invention which is defined by the appended claims.

What is claimed is:

1. An extract of Vitis thunbergii var. taiwaniana, which is prepared by the following steps: extracting one or more tissues of Vitis thunbergii var. taiwaniana with 10 wt % to 100 wt % of alcohol solution or water to obtain an extract solution, and then drying said extract solution.

2. The extract of claim 1, wherein said alcohol is methanol, ethanol, propanol or isopropanol.

3. The extract of claim 1, which is extracted with water, and said extract solution is further processed by the following steps: gradient-eluting said extract solution by using porous adsorbent resin as stationary phase and water to an alcohol as mobile phase, and then collecting eluates eluted by 50 wt % or more of alcohol solution.

4. The extract of claim 1, wherein said one or more tissues of Vitis thunbergii var. taiwaniana is root or stem.

5. A use of the extract of claim 1 in preparing a medicine for blood pressure regulation.

6. A pharmaceutical composition for hypertension prevention or treatment, which contains an effective amount of the extract of claim 1.

7. A method for isolating (+)-vitisin A or ampelopsin C from Vitis thunbergii var. taiwaniana, which includes the steps of:
(a) dissolving the extract of claim 1 in the alcohol solution to obtain a dissolved solution;
(b) gradient-eluting the dissolved solution of step (a) by using dextran gel as stationary phase and an alcohol to a ketone as mobile phase, and then collecting first batch of eluates;
(c) gradient-eluting the first batch of eluates from step (b) by using amorphous silicon as stationary phase and a C1 to C3 organic acid to acetonitrile as mobile phase, and then collecting second batch of eluates; and
(d) identifying (+)-vitisin A or ampelopsin C in the second batch of eluates obtained from the step (c).

8. The method of claim 7, wherein said alcohol is methanol, ethanol, propanol or isopropanol.

9. The method of claim 7, wherein said ketone is acetone.

10. The method of claim 7, wherein said C1 to C3 organic acid is trifluoroacetic acid.

11. A use of (+)-vitisin A or ampelopsin C in hypertension prevention or treatment.

12. A product for hypertension prevention or treatment, which contains one or more tissues of Vitis thunbergii var. taiwaniana, an extract of Vitis thunbergii var. taiwaniana, (+)-vitisin A or ampelopsin C.

13. The product of claim 12, wherein said product is in form of drink, tablet or instant drink powder.
